(19) United States
(12) Patent Application Publication
(10) Pub. No.: US 2009/0222088 A1
(43) Pub. Date: Sep. 3, 2009

(54) Secondary Amine Containing Nitric Oxide Releasing Polymer Composition

(75) Inventors: Mingfei Chen, Santa Rosa, CA (US); Peiwen Cheng, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/040,503

(22) Filed: Feb. 29, 2008

(51) Int. Cl. A61F 2/02; C08F 20/06; A61B 17/08; C07C 211/22

(52) U.S. Cl. 623/11.11; 526/317.1; 564/511; 606/151

(57) ABSTRACT

Disclosed herein are polymers used to coat or form implantable medical devices. The polymers comprise secondary amines useful in binding nitric oxide (NO). After diazeniumdiolation, the polymers can sustain controlled release of NO. In one embodiment, the secondary amines are linked to a functionalized dendrimer. In another embodiment, secondary amines are chelated with copper (II) which in turn serve as a catalyst for NO production.

### FIELD OF THE INVENTION

The present invention relates to nitric oxide (NO) donating polymers for fabricating and coating medical devices.

### BACKGROUND OF THE INVENTION

Nitric oxide (NO) is a simple diatomic molecule that plays a diverse and complex role in cellular physiology. Less than 25 years ago NO was primarily considered a smog component formed during the combustion of fossil fuels mixed with air. However, as a result of the pioneering work of Ferid Murad et al. it is now known that NO is a powerful signaling compound and cytotoxic/cytostatic agent found in nearly every tissue including endothelial cells, neural cells and macrophages. Mammalian cells synthesize NO using a two step enzymatic process that oxidizes L-arginine to N-ω-hydroxy-L-arginine, which is then converted into L-citrulline and an uncharged NO free radical. Three different nitric oxide synthase enzymes regulate NO production. Neuronal nitric oxide synthase (NOSI, or nNOS) is formed within neuronal tissue and plays an essential role in neurotransmission; endothelial nitric oxide synthase (NOS3 or eNOS), is secreted by endothelial cells and induces vasodilatation; inducible nitric oxide synthase (NOS2 or iNOS) is principally found in macrophages, hepatocytes and chondrocytes and is associated with immune cytotoxicity.

Neuronal NOS and eNOS are constitutive enzymes that regulate the rapid, short-term release of small amounts of NO. In these minute amounts NO activates guanylate cyclase which elevates cyclic guanosine monophosphate (cGMP) concentrations which in turn increase intracellular Ca²⁺ levels. Increased intracellular Ca²⁺ concentrations result in smooth muscle relaxation which accounts for NO's vasodilating effects. Inducible NOS is responsible for the sustained release of larger amounts of NO and is activated by extracellular factors including endotoxins and cytokines. These higher NO levels play a key role in cellular immunity.

Medical research is rapidly discovering therapeutic applications for NO including the fields of vascular surgery and interventional cardiology. Procedures used to clear blocked arteries such as percutaneous transluminal coronary angioplasty (PTCA) (also known as balloon angioplasty) and atherectomy and/or stent placement can result in vessel wall injury at the site of balloon expansion or stent deployment. In response to this injury a complex multi-factorial process known as restenosis can occur whereby the previously opened vessel lumen narrows and becomes re-occluded. Restenosis is initiated when thrombocytes (platelets) migrating to the injury site release mitogens into the injured endothelium. Thrombocytes begin to aggregate and adhere to the injury site initiating thrombogenesis, or clot formation. As a result, the previously opened lumen begins to narrow as thrombocytes and fibrin collect on the vessel wall. In a more frequently encountered mechanism of restenosis, the mitogens secreted by activated thrombocytes adhering to the vessel wall stimulate overproliferation of vascular smooth muscle cells during the healing process, restricting or occluding the injured vessel lumen. The resulting neointimal hyperplasia is the major cause of a stent restenosis.

Recently, NO has been shown to significantly reduce thrombocyte aggregation and adhesion; this combined with NO's directly cytotoxic/cytostatic properties may significantly reduce vascular smooth muscle cell proliferation and help prevent restenosis. Thrombocyte aggregation occurs within minutes following the initial vascular insult and once the cascade of events leading to restenosis is initiated, irreparable damage can result. Moreover, the risk of thrombogenesis and restenosis persists until the endothelium lining the vessel lumen has been repaired. Therefore, it is essential that NO, or any anti-restenotic agent, reach the injury site immediately.

One approach for providing a therapeutic level of NO at an injury site is to increase systemic NO levels prophylactically. This can be accomplished by stimulating endogenous NO production or using exogenous NO sources. Methods to regulate endogenous NO release have primarily focused on activation of synthetic pathways using excess amounts of NO precursors like L-arginine, or increasing expression of nitric oxide synthase (NOS) using gene therapy. U.S. Pat. Nos. 5,945,452, 5,891,459 and 5,428,070 describe sustained NO elevation using orally administrated L-arginine and/or L-lysine. However, these methods have not been proven effective in preventing restenosis. Regulating endogenously expressed NO using gene therapy techniques remains highly experimental and has not yet proven safe and effective. U.S. Pat. Nos. 5,268,465, 5,468,630 and 5,658,565, describe various gene therapy approaches.

Exogenous NO sources such as pure NO gas are highly toxic, short-lived and relatively insoluble in physiological fluids. Consequently, systemic exogenous NO delivery is generally accomplished using organic nitrate prodrugs such as nitroglycerin tablets, intravenous suspensions, sprays and transdermal patches. The human body rapidly converts nitroglycerin into NO; however, enzyme levels and co-factors required to activate the prodrug are rapidly depleted, resulting in drug tolerance. Moreover, systemic NO administration can have devastating side effects including hypotension and free radical cell damage. Therefore, using organic nitrate prodrugs to maintain systemic anti-restenotic therapeutic blood levels is not currently possible.

Therefore, considerable attention has been focused on localized, or site specific, NO delivery to ameliorate the disadvantages associated with systemic prophylaxis. Implantable medical devices and/or local gene therapy techniques including medical devices coated with NO-releasing compounds, or vectors that deliver NOS genes to target cells, have been evaluated. Like their systemic counterparts, gene therapy techniques for the localized NO delivery have not been proven safe and effective. There are still significant technical hurdles and safety concerns that must be overcome before site-specific NOS gene delivery will become a reality.

However, significant progress has been made in the field of localized exogenous NO application. To be effective at preventing restenosis an inhibitory therapeutic such as NO must be administered for a sustained period at therapeutic levels. Consequently, any NO-releasing medical device used to treat restenosis must be suitable for implantation. An ideal candidate device is the vascular stent. Therefore, a stent that safely provides therapeutically effective amounts of NO to a precise location would represent a significant advance in restenosis treatment and prevention.

Nitric oxide-releasing compounds suitable for in vivo applications have been developed by a number of investigators. It has been demonstrated that nitric oxide gas can be reacted with amines, for example, diethylamine, to form NO-releasing anions having the following general formula R—R′N—N(O)NO. Salts of these compounds could spontaneously decompose and release NO in solution.

Nitric oxide-releasing compounds with sufficient stability at body temperatures to be useful as therapeutics were ultimately developed by Keefer et al. as described in U.S. Pat. Nos. 4,954,526, 5,039,705, 5,155,137, 5,212,204, 5,250,550, 5,366,997, 5,405,919, 5,525,357 and 5,650,447, all of which are herein incorporated by reference.

The in vivo half-life of NO, however, is limited, causing difficulties in delivering NO to the intended area. Therefore NO-releasing compounds which can produce extended release of NO are needed. Several exemplary NO-releasing compounds have been developed for this purpose, including for example a NO donating aspirin derivative, amyl nitrite and isosorbide dinitrate. Additionally, biocompatible polymers having NO adducts (see, for example, U.S. Patent Publications 2006/0008529 and 2004/0037836) and which release NO in a controlled manner have been reported.

Secondary amines have the ability to bind two NO molecules and release them in an aqueous environment. The general structure of exemplary secondary amines capable of binding two NO molecules is depicted in Formula 1, referred to hereinafter a diazeniumdiolate, (wherein M is a counterion, and can be a metal, with the appropriate charge, or a proton and wherein R is a generic notation for organic and inorganic chemical groups). Exposing secondary amines to basic conditions while incorporating NO gas under high pressure leads to the formation of diazeniumdiolates.

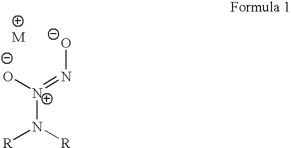

### SUMMARY OF THE INVENTION

Provided herein are nitric oxide releasing polymers bearing multiple amines, which are diazeniumdiolated, in their functional groups as well as chelated copper ions, which catalyze the synthesis of nitric oxide from nitrogen sources in the surrounding physiological atmosphere.

In one embodiment, a nitric oxide (NO)-donating polymer is described comprising a diazeniumdiolate binding scaffolding; said scaffolding comprising monomers with at least one diazeniumdiolate binding site; wherein said binding sites comprise secondary amines. In another embodiment, the NO-donating polymer further comprises multiple amine chelated copper ions.

In one embodiment, the NO donating polymer comprises at least one monomer selected from the group consisting of methyl methacrylate, ethyl methacrylate, propyl methacrylate, butyl methacrylate, pentyl methacrylate, hexyl methacrylate, 2-ethylhexyl methacrylate, octyl methacrylate, lauryl methacrylate, 2-ethoxyethyl methacrylate, 2-hydroxyethyl methacrylate, and hydroxypropyl methacrylate, methyl acrylate, ethyl acrylate, propyl acrylate, butyl acrylate, pentyl acrylate, hexyl acrylate, 2-ethylhexyl acrylate, octyl acrylate and lauryl acrylate, 2-hydroxyethyl acrylate, and hydroxypropyl acrylate. In one embodiment, the acrylate monomer is glycidyl methacrylate which has epoxide side chain. Non-acrylate monomers include, but are not limited to, ε-caprolactone, polyethylene glycol (PEG), trimethylene carbonate, lactide, glycolide, p-dioxanone, N-acetyl caprolactone, cyclohexyl caprolactone, 4-tert-butyl caprolactone, the caprolactone of Formula 4, and their derivatives and combinations thereof.

In one embodiment, the NO-donating polymer comprises the coating of an implantable medical device. In another embodiment, the NO-donating polymer comprises an implantable medical device. In yet another embodiment, the medical device is selected from the group consisting of vascular stents, stent grafts, urethral stents, bile duct stents, catheters, guide wires, pacemaker leads, bone screws, sutures and prosthetic heart valves.

In one embodiment, the NO-donating polymer comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

In one embodiment, the secondary amines are on a dendrimer. In another embodiment, the secondary amines can be coordinated with a copper ligand.

In one embodiment, a medical device is described comprising a diazeniumdiolate binding scaffolding; a polymer comprises said scaffolding having monomers with at least one diazeniumdiolate binding site; wherein said binding sites comprise secondary amines; and wherein a medical device comprises said scaffolding.

In one embodiment, the medical device further comprises multiple amine chelated copper ions. In one embodiment, the polymer comprises at said polymer comprises at least one monomer selected from the group consisting of methyl methacrylate, ethyl methacrylate, propyl methacrylate, butyl methacrylate, pentyl methacrylate, hexyl methacrylate, 2-ethylhexyl methacrylate, octyl methacrylate, lauryl methacrylate, 2-ethoxyethyl methacrylate, 2-hydroxyethyl methacrylate and hydroxypropyl methacrylate, methyl acrylate, ethyl acrylate, propyl acrylate, butyl acrylate, pentyl acrylate, hexyl acrylate, 2-ethylhexyl acrylate, octyl acrylate and lauryl acrylate, 2-hydroxyethyl acrylate, and hydroxypropyl acrylate. In one embodiment, the acrylate monomer is glycidyl methacrylate which has epoxide side chains. Non-acrylate monomers include, but are not limited to, ε-caprolactone, polyethylene glycol (PEG), trimethylene carbonate, lactide, glycolide, p-dioxanone, N-acetyl caprolactone, cyclohexyl caprolactone, 4-tert-butyl caprolactone, the caprolactone of Formula 4, and their derivatives and combinations thereof.

In one embodiment, the polymer comprises the coating of an implantable medical device. In another embodiment, the polymer comprises an implantable medical device. In one embodiment, the medical device is selected from the group consisting of vascular stents, stent grafts, urethral stents, bile duct stents, catheters, guide wires, pacemaker leads, bone screws, sutures and prosthetic heart valves.

In one embodiment, the polymer comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

In one embodiment, the secondary amines are on a dendrimer. In another embodiment, the secondary amines can be coordinated with a copper ligand.

In one embodiment, a method is described of forming a NO-donating polymer comprising a NO-donating scaffolding comprising a) providing a polymer with side chains having at least one epoxide; b) reacting an amine with said epoxide wherein said amine comprises a NO-donating scaffolding thereby forming a NO-donating scaffolded polymer; and c) loading said scaffolded polymer with NO thereby forming an NO-donating polymer.

In one embodiment, the method of forming a NO-donating polymer according to claim 19, wherein said method further comprises the step of d) forming at least a portion of a medical device with said NO-donating polymer.

In one embodiment, the amine is selected from N-methylethylenediamine, N-methylpropylylenediamine, N-methylbutylenediamine, N-ethylethylenediamine, N-ethylpropylylenediamine, N-ethylbutylenediamine, N-benzylethylenediamine, N-benzylpropylylenediamine, N-benzylbutylenediamine, N-propylethylenediamine, N-propylpropylylenediamine, N-propylbutylenediamine, ethylenediamine, diethylenetriamine, triethylenetetramine, tetraethylenepentaamine, pentaethylenehexamine, and hexaethyleneheptaamine.

### DEFINITION OF TERMS

Backbone: As used herein, “backbone” refers to the main chain of a polymer or copolymer of the present invention. A “polyester backbone” as used herein refers to the main chain of a biodegradable polymer comprising ester linkages.
Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.
Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the polymers described herein.
Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.
Copolymer: As used herein, a “copolymer” is a macromolecule produced by the simultaneous chain addition polymerization of two or more dissimilar units such as monomers. Copolymers include bipolymers (two dissimilar units), terpolymers (three dissimilar units), etc.
Controlled release: As used herein, “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.
Dendrimer: As used herein, “dendrimer” shall refer to synthetic macromolecules comprised of regular branched repeat units in layers emanating radially from a point-like core. Formula 13 is a non-limiting example, and one skilled in the art will appreciate that there are numerous examples of dendrimers that can be used. Dendrimers are branched structures with multivalent surfaces. The size of a dendrimer varies greatly depending on its type and the functional groups on the surface. An exemplary dendrimer can be a polypropylenimine dendrimer due to its many functional amine groups on the surface. However, other dendrimers of interest in the current invention include, but are not limited to polyether, polyester and polyamide.
Diazeniumdiolate: As used herein, “diazeniumdiolate” refers to a class of nitric oxide donating molecules, also referred to as NONOates (1-substituted diazen-1-ium-1,2-diolates) are chemical species that carry the [N(O)NO]— functional group and release nitric oxide (NO) molecules under physiological conditions at a predictable rate. Furthermore, “diazeniumdiolated” or “diazeniumdiolation” refers to molecules having diazeniumdiolate groups or the process of adding such groups to a polymer.
Glass Transition Temperature (T_{g}): As used herein, “glass transition temperature” or “T_{g}” refers to a temperature wherein a polymer structurally transitions from a elastic pliable state to a rigid and brittle state.
Glycidyl Methacrylate: As used herein, glycidyl methacrylate refers to a molecule having the general structure:

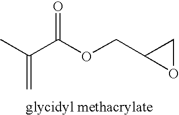

Mₙ: As used herein, Mₙ refers to number-average molecular weight. Mathematically it is represented by the following formula:

$${M}_{n} = {\sum }_{i} {N}_{i} {M}_{i} / {\sum }_{i} {N}_{i} ,$$

wherein the Nᵢ is the number of moles whose weight is M.

M_{w}: As used herein, M_{w} refers to weight average molecular weight that is the average weight that a given polymer may have. Mathematically it is represented by the following formula:

$${M}_{w} = {\sum }_{i} {N}_{i} {M}_{i}^{2} / {\sum }_{i} {N}_{i} {M}_{i} ,$$

wherein Nᵢ is the number of molecules whose weight is Mᵢ.

### DETAILED DESCRIPTION OF THE INVENTION

Described herein are nitric oxide (NO)-releasing polymers bearing at least one secondary amine per functional monomer. The secondary amine can bind nitric oxide to form diazeniumdiolate group. Having multiple secondary amines per monomer unit allows for loading of multiple diazeniumdiolates per monomer unit. Provided herein is a means for creating a scaffolding on a polymer on which NO may be loaded.

Polymers containing secondary amines therein have been synthesized and diazeniumdiolated. Increasing the numbers of secondary amines in the polymers provides for increased NO loading, more stable diazeniumdiolates and enhances the prospect of more finely tuned controlled release. Applicants have determined that biocompatible polymers based on epoxide-opening reactions wherein the completed polymers may encompass a scaffolding of secondary amines. The resulting NO-donating polymers are suitable for fabricating and coating medical devices.

In one embodiment, multiple amines can be added to a polymer by binding an amine terminated dendrimer to the monomers or to the polymers directly. Secondary amine terminated dendrimers can be reacted, for example, with an epoxide containing monomer to provide a dendrimer linked to the polymer wherein the dendrimer has a scaffolding of secondary amines capable of binding NO. In one embodiment, the dendrimer can be added to the polymer via epoxide opening by a secondary amine giving a tertiary amine linked dendrimer.

In another embodiment, amine based pockets that bind copper are disclosed. It has been found that copper ions catalyze the formation of nitric oxide (NO) from the surrounding physiological atmosphere. In one embodiment, polymers with small amounts of amine sequestered copper ions that catalyze the synthesis of NO from nitrogen sources in the physiological atmosphere are described. The copper ions in the polymers can catalyze the synthesis of NO and continuously provide an appropriate dose of NO.

Epoxide-derived (NO) donating polymers suitable for fabricating and coating medical devices are described herein. More specifically, the present description provides polymers comprising monomer side chains having at least one secondary amine per functional monomer that can be diazeniumdiolated to release or donate NO controllably in a physiological environment. Furthermore, a method for the synthesis of epoxide-derived polymers comprising secondary amines is disclosed.

The polymers comprise homopolymers and copolymers. The homopolymers consist of monomer units comprising at least one secondary amine group on each side chain. The polymers include, but are not limited to, acrylates polyesters, polycarbonates, polyethers, polyurethanes, and other biostable and biodegradable polymers.

Monomers suitable for use in the methods include monomers having the general structure of Formula 2

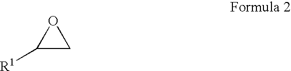

wherein R¹ is a polymerizable moiety including, but not limited to acrylates, lactones, C₂ to C₂₀ alkenyl, and C₂ to C₂₀ alkynyl.

Polymer backbones suitable for use in the present methods include backbones selected from the group consisting of polyethers, polyesters, acrylates and derivatives and combinations thereof.

The acrylate polymers comprise acrylic monomers including, but not limited to, methyl methacrylate, ethyl methacrylate, propyl methacrylate, methyl, butyl methacrylate, pentyl methacrylate, hexyl methacrylate, 2-ethylhexyl methacrylate, octyl methacrylate, lauryl methacrylate and 2-ethoxyethyl methacrylate, 2-hydroxyethyl methacrylate, hydroxypropyl methacrylate, methyl acrylate, ethyl acrylate, propyl acrylate, butyl acrylate, pentyl acrylate, hexyl acrylate and 2-ethylhexyl acrylate, octyl acrylate, lauryl acrylate, 2-hydroxyethyl acrylate and hydroxypropyl acrylate. In one embodiment, the acrylate monomer is glycidyl methacrylate which has an epoxide side chain. Non-acrylate monomers include, but are not limited to, ε-caprolactone, polyethylene glycol (PEG), trimethylene carbonate, lactide, glycolide, p-dioxanone, N-acetyl caprolactone, cyclohexyl caprolactone, 4-tert-butyl caprolactone, the caprolactone of Formula 4, and their derivatives.

The polymers are comprised of at least one monomer having at least one secondary amine group on each side chain. The secondary amine groups can be introduced either before (Reaction 1 in Scheme 1 producing Formula 3) or after (Reaction 2 in Scheme 1) monomer polymerization. In one embodiment, the secondary amines may be introduced to the polymer through reaction of a molecule containing an amine with the epoxide group on either monomers or polymers. In one embodiment, the secondary amines may be introduced through nucleophilic or electrophilic epoxide-opening reactions on either monomers or polymers. The general reaction is presented in Scheme 1, wherein R¹ is a polymerizable moiety including, but not limited to acrylates, lactones, C₂ to C₂₀ alkenyl, C₂ to C₂₀ alkynyl; R² and R³ are independently hydrogen, a C₁ to C₁₀ straight chain alkyl, C₃ to C₈ cycloalkyl, C₂ to C₂₀ alkenyl, C₂ to C₂₀ alkynyl, C₂ to C₁₄ heteroatom substituted alkyl, C₂ to C₁₄ heteroatom substituted cycloalkyl, C₁ to C₁₂ multiple amine-containing hydrocarbons, C₄ to C₁₀ substituted aryls, C₄ to C₁₀ substituted heteroatom substituted heteroaryls, or a dendrimer. In the case that R² or R³ is hydrogen then the reactant molecule is a primary amine. Exemplary C₁-C₁₀ multiple amine-containing hydrocarbons include, but are not limited to, N-methylethylenediamine, N-methylpropylylenediamine, N-methylbutylenediamine, N-ethylethylenediamine, N-ethylpropylylenediamine, N-ethylbutylenediamine, N-benzylethylenediamine, N-benzylpropylylenediamine, N-benzylbutylenediamine, N-propylethylenediamine, N-propylpropylylenediamine, N-propylbutylenediamine, ethylenediamine, diethylenetriamine, triethylenetetramine, tetraethylenepentaamine, pentaethylenehexamine and hexaethyleneheptaamine.

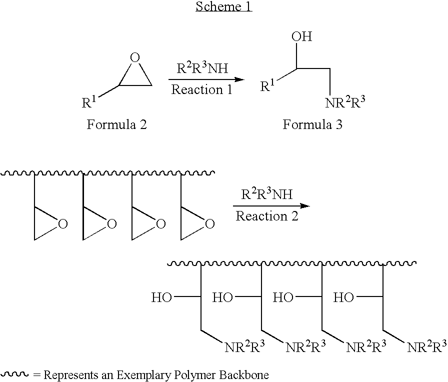

The epoxides in the polymer side chains are synthesized by reactions including dehydration reactions, oxidization of alkenes, and ring closing reactions. An exemplary monomer having an epoxide-containing side chain is glycidyl methacrylate. Methods of synthesizing the epoxide side chains on monomers to prepare the monomers for polymerization are also disclosed. In one embodiment, alkene-containing polymerizable monomers are treated with dimethyldioxirane to yield the epoxide-containing side chain. In another embodiment, the alkene-containing monomer is 2-allyl caprolactone (Formula 4). The epoxidation of the alkene-containing monomers are performed either after polymerization or before polymerization of the monomers.

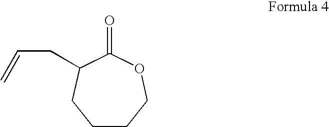

The alkene-containing monomers can be homopolymerized or copolymerized with different monomers. In one embodiment, 2-allyl caprolactone (Formula 4) is copolymerized with other lactones such as, but not limited to, glycolide, lactide, and other biocompatible lactones. The resulting copolymer is then epoxidized. The epoxidation reaction on the alkene-containing monomers or the alkene-containing polymers can be carried out with a number of reagents such as, but not limited to, dimethyldioxirane, mCPBA, metal oxides, peroxides, peracids, cyclic peroxides, and derivatives thereof. In another embodiment the 2-allyl caprolactone is homopolymerized and the resultant polymer then epoxidated. Once the polymers having epoxide-containing side chains are synthesized, they can be treated with secondary amines to yield polymers having secondary amine side chains (as illustrated in Scheme 1). The resulting polymers having secondary side chains as depicted in the products of Reaction 2 (Scheme 1) can also be considered as amino alcohols.

In one embodiment, the polymers comprise at least one secondary amine per amine-bearing, functional, monomer unit. The secondary amines are introduced through nucleophilic attack of the amines on an electrophilic moiety on the polymerized monomer unit. The reactions introducing the amines through nucleophilic attack are optionally catalyzed. Catalysts useful in synthesizing the polymers include but are not limited to LiClO₄, mineral acids, Bronsted acids, ion exchange resins, zeolites, oxophilic metals, proton sponges, buffer solutions, alkali earth metals, alkaline earth metals, transition metals, and organometallic compounds. In one embodiment depicted in Formula 5, an amine is introduced on a polymer derived from glycidyl methacrylate and a methacrylate through nucleophilic attack on the epoxide. In Formulae 5 and 6, R³ is a C₁ to C₁₀ straight chain alkyl, C₅ to C₁₀ cycloalkyl, alkoxy substituted C₂ to C₁₀ alkyl, heteroatom substituted C₂ to C₁₀ alkyl, polyethylene glycol (PEG) (Reaction 3), or a dendrimer. R¹ and R² are independently hydrogen a C₁ to C₁₀ straight chain alkyl, C₃ to C₈ cycloalkyl, C₂ to C₂₀ alkenyl, C₂ to C₂₀ alkynyl, C₂ to C₁₄ heteroatom substituted alkyl, C₂ to C₁₄ heteroatom substituted cycloalkyl, C₁ to C₁₂ multiple amine-containing hydrocarbons, C₄ to C₁₀ substituted aryl, or C₄ to C₁₀ substituted heteroatom substituted heteroaryl. In the case either R¹ or R² is hydrogen, the reactant is a primary amine.

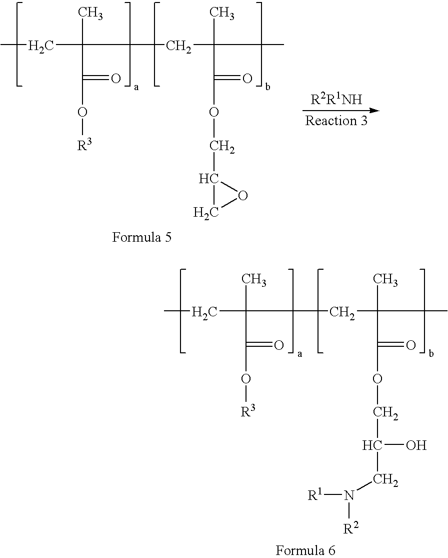

In one embodiment, the a and b units of Formulae 5 and 6 are individually integers from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000. In additional embodiments, b is an integer ranging from 10 to 20,000; from 50 to 15,000; from 100 to 10,000; from 200 to 5,000; from 500 to 4,000; from 700 to 3,000; or from 1000 to 2000.

In one embodiment, the side chains can be synthetically fine tuned to provide controlled release of NO by choosing the appropriate amines for nucleophilic attack on the precursor polymers.

In one embodiment, the side chains are also impregnated with a small amount of copper wherein the copper is sequestered by the multiple amines present in the polymer. In another embodiment, NO donating amines are contained within the molecule added to the polymer. In such a case, copper is optionally used based on the amine groups and whether or not they require copper chelation.

Non-acrylate polymers include polyesters, polycarbonates, polyethers, polyurethanes, and other biostable or biodegradable polymers. In one embodiment, the NO donating polymer is a polyester of Formula 9, wherein n is an integer from 1 to 4, m is an integer from 1 to 20,000 and R¹ is hydrogen. The polyester of Formula 9 is synthesized from the epoxide of Formula 8 through a standard ring opening reaction with a primary amine. In Formula 9, R¹ and R² are independently hydrogen, a C₁ to C₁₀ straight chain alkyl, C₃ to C₈ cycloalkyl, C₂ to C₂₀ alkenyl, C₂ to C₂₀ alkynyl, C₂ to C₁₄ heteroatom substituted alkyl, C₂ to C₁₄ heteroatom substituted cycloalkyl, C₁ to C₁₂ multiple amine-containing hydrocarbons, C₄ to C₁₀ substituted aryl, C₄ to C₁₀ substituted heteroatom substituted heteroaryl, or a dendrimer.

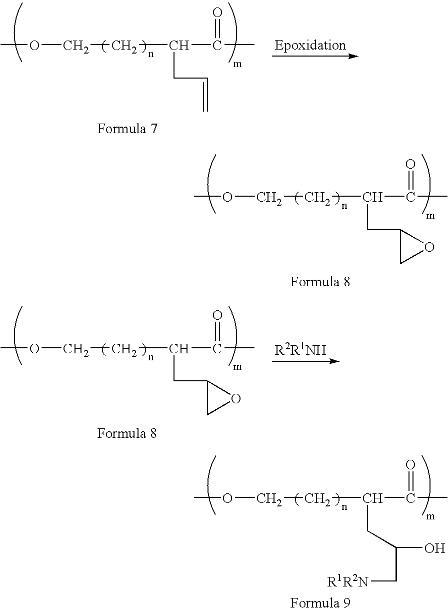

In one embodiment of Formulae 7, 8 and 9, m is an integer ranging from 1 to 20,000. In additional embodiments, m is an integer ranging from 10 to 19,000, from 200 to 17,000, from 400 to 15,000, from 500 to 14,000, from 600 to 13,000, from 700 to 12,000, from 800 to 11,000, from 900 to 12,000, from 1,000 to 11,000, from 1,100 to 10,000, from 1,200 to 9,000, from 1,300 to 8,000, from 1,400 to 7,000, from 1,500 to 6,000, from 1,600 to 5,000, from 1,600 to 4,000, from 1,700 to 3,000, from 1,800 to 2,000 or from 1,900 to 1,950. In another embodiment of Formulae 7, 8 and 9 of the present invention, n is an integer ranging from 1 to 4. In additional embodiments, n is 2 or 3.

The non-acrylic polymers are not limited to homopolymers. In one embodiment, copolymers of polyethers and polyesters are synthesized according to Formula 10 which undergoes the reactions described above to form the epoxide of Formula 11. The epoxide of Formula 11 is then treated with a primary amine to yield the polymer of Formula 12. In Formula 12, R¹ and R² are independently hydrogen, C₁ to C₁₀ straight chain alkyl, C₃ to C₈ cycloalkyl, C₁ to C₁₂ multiple amine-containing hydrocarbons, C₂ to C₂₀ alkenyl, C₂ to C₂₀ alkynyl, C₂ to C₁₄ heteroatom substituted alkyl, C₂ to C₁₄ heteroatom substituted cycloalkyl, C₄ to C₁₀ substituted aryl, C₄ to C₁₀ substituted heteroatom substituted heteroaryl, or a dendrimer. In Formula 12, n is an integer from 1 to 4, m is an integer from 1 to 20,000 and f is an integer from 1 to 20,000.

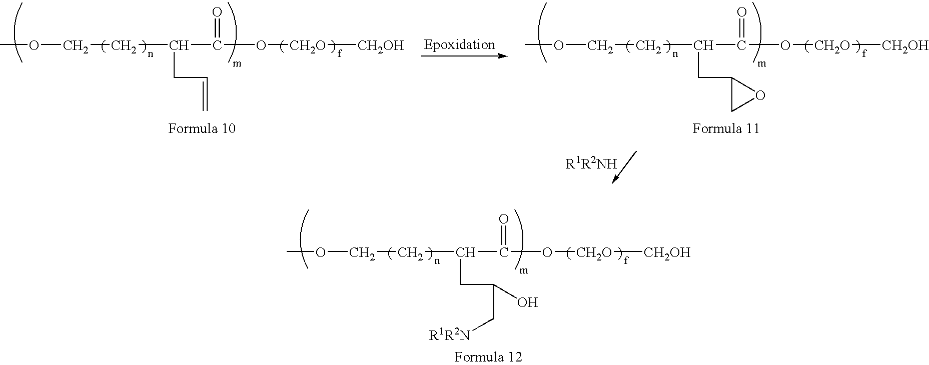

In one embodiment of Formulae 10, 11 and 12, m and f are individually integers from 1 to 20,000. In additional embodiments, m is an integer ranging from 10 to 19,000, from 200 to 17,000, from 400 to 15,000, from 500 to 14,000, from 600 to 13,000, from 700 to 12,000, from 800 to 11,000, from 900 to 12,000, from 1,000 to 11,000, from 1,100 to 10,000, from 1,200 to 9,000, from 1,300 to 8,000, from 1,400 to 7,000, from 1,500 to 6,000, from 1,600 to 5,000, from 1,600 to 4,000, from 1,700 to 3,000, from 1,800 to 2,000 or from 1,900 to 1,950. In additional embodiments, f is an integer ranging from 10 to 19,000, from 200 to 17,000, from 400 to 15,000, from 500 to 14,000, from 600 to 13,000, from 700 to 12,000, from 800 to 11,000, from 900 to 12,000, from 1,000 to 11,000, from 1,100 to 10,000, from 1,200 to 9,000, from 1,300 to 8,000, from 1,400 to 7,000, from 1,500 to 6,000, from 1,600 to 5,000, from 1,600 to 4,000, from 1,700 to 3,000, from 1,800 to 2,000 or from 1,900 to 1,950. In another embodiment of Formulae 10, 11 and 12, n is an integer ranging from 1 to 4. In additional embodiments, n is 2 or 3.

In one embodiment, secondary amines can be provided on dendrimers similar that that of formula 13. Dendrimers are branched structures with multivalent surfaces. The size of a dendrimer varies greatly depending on its type and the functional groups on the surface. An exemplary dendrimer can be a polypropylenimine dendrimer due to its many functional amine groups on the surface (Formula 13). The size of the exemplary dendrimer is G3 and is can bind up to 32 moles of NO.

Dendrimers are ideal scaffolding for NO donation because a dendrimer properly modified may accommodate large amounts of NO; the large amount of NO can be per monomer unit. In reaction 3, the terminal amines may be primary amines if R¹ or R² is hydrogen. However, each R can be independently selected from the group consisting of hydrogen, C₁ to C₁₀ straight chain alkyl, C₅ to C₁₀ cycloalkyl, alkoxy substituted C₂ to C₁₀ alkyl, heteroatom substituted C₂ to C₁₀ alkyl or polyethylene glycol (PEG) (Reaction 3). If R is not hydrogen, the functional group will be a secondary amine.

In Formula 14, R² is hydrogen, a C₁ to C₁₀ straight chain alkyl, C₃ to C₈ cycloalkyl, C₂ to C₂₀ alkenyl, C₂ to C₂₀ alkynyl, C₂ to C₁₄ heteroatom substituted alkyl, C₂ to C₁₄ heteroatom substituted cycloalkyl, C₁ to C₁₂ multiple amine-containing hydrocarbons, C₄ to C₁₀ substituted aryl, or C₄ to C₁₀ substituted heteroatom substituted heteroaryl. If R² is not hydrogen, then the amine is secondary.

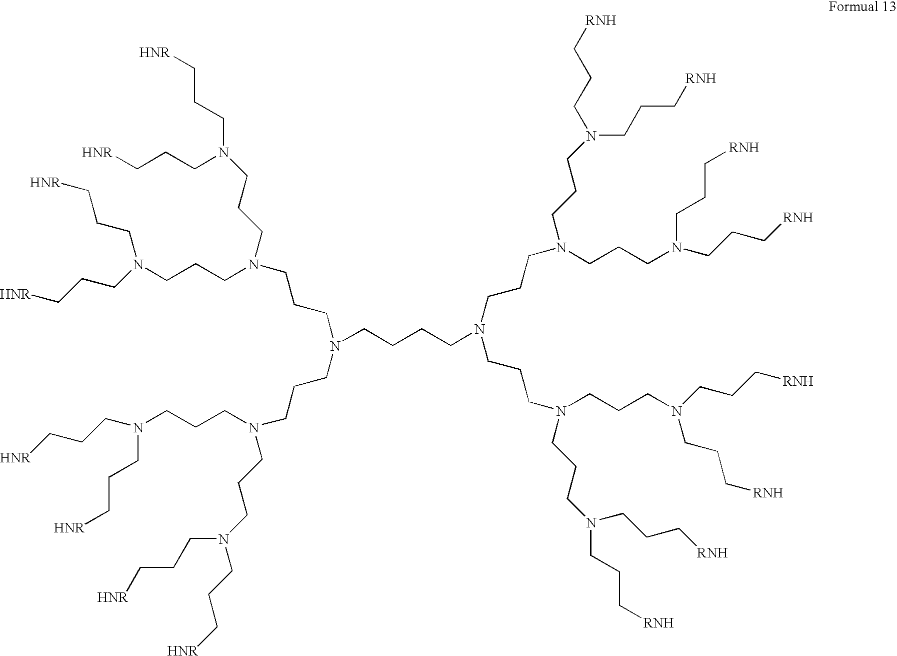

In one embodiment, if R² is hydrogen, the primary amines may be converted to secondary amines through the following reaction.

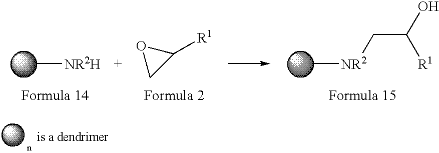

In the above reaction, if R² is hydrogen, primary amine groups of a dendrimer are reacted with a epoxide via a nucleophilic or electrophilic epoxide-opening reaction. In the above reaction, R¹ is a C₁ to C₁₀ straight chain alkyl, C₃ to C₈ cycloalkyl, C₂ to C₂₀ alkenyl, C₂ to C₂₀ alkynyl, C₂ to C₁₄ heteroatom substituted alkyl, C₂ to C₁₄ heteroatom substituted cycloalkyl, C₁ to C₁₂ multiple amine-containing hydrocarbons, C₄ to C₁₀ substituted aryls, or C₄ to C₁₀ substituted heteroatom substituted heteroaryls. The product, Formula 15, is a secondary amine functionalized dendrimer (in the case R² is hydrogen) which can be reacted with a polymerizable monomer or more preferably a preassembled polymer.

In one embodiment, the polymer contains monomers with epoxide containing side chains. The secondary amine functionalized dendrimer can be reacted with an epoxide on a polymer side chain according to the following reaction. The resulting polymer possesses dendrimers with scaffolding capable of loading NO at a much greater NO/monomer than a monomer with a single amine side chain.

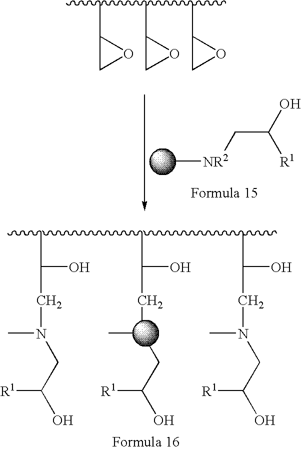

In one embodiment, polymers are provided that comprise small amounts of copper ions, wherein the copper ions are sequestered by multiple amine residues in the side chains of the polymer. The copper ion catalyzes the production of NO from nitrogen sources in the surrounding physiological atmosphere therefore providing a sufficient amount of NO in the blood stream. The copper ions can be incorporated into the polymer in one predominant way. In one embodiment the polymers are dissolved, in a suitable solvent, in the presence of copper (II) chloride. A change in the color of the solution indicates the binding of the copper by the amine residues.

In one embodiment, NO releasing polymers are provided wherein the polymers comprise secondary amines chelating copper ions. The copper ions catalyze the synthesis of NO, providing a constant NO supply to the affected area. In one embodiment the polymers of Formula 6 are impregnated with copper. In another embodiment the polymers of Formula 9 are impregnated with copper. In still another embodiment the polymers of Formula 12 are impregnated with copper. The above mentioned polymers are used to coat and fabricate medical devices.

Not all of the amine residues will sequester the copper ions in the polymer, some amines will be free to be diazeniumdiolated with the traditional basified pressurizing methods. Consequently, the copper impregnated polymers of the present invention have the ability to not only catalyze NO synthesis in vivo, but they also have the ability to be diazeniumdiolated prior to being placed inside the body. This system provides for a two-pronged method releasing NO.

The physical properties of the polymers can be fine tuned so that the polymers can optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphlicity. In one embodiment, the T_{g} of the polymers range from about −10° C. to about 85° C. In still another embodiment, the PDI of the polymers range from about 1.35 to about 4. In another embodiment of the present invention, the T_{g} of the polymers ranges form about 0° C. to about 40° C. In still another embodiment, the PDI of the polymers range from about 1.5 to about 2.5.

Implantable medical devices suitable for coating with the epoxide-derived NO-donating polymers include, but are not limited to, vascular stents, stent grafts, urethral stents, bile duct stents, catheters, guide wires, pacemaker leads, bone screws, sutures and prosthetic heart valves. The polymers are suitable for fabricating implantable medical devices. Medical devices which can be manufactured from the epoxide-derived NO-donating polymers include, but are not limited to, vascular stents, stent grafts, urethral stents, bile duct stents, catheters, guide wires, pacemaker leads, bone screws, sutures and prosthetic heart valves.

The polymeric coatings are intended for medical devices deployed in a hemodynamic environment and possess excellent adhesive properties. That is, the coating must be stably linked to the medical device surface. Many different materials can be used to fabricate the implantable medical devices including, but not limited to, stainless steel, nitinol, aluminum, chromium, titanium, gold, cobalt, ceramics, and a wide range of synthetic polymeric and natural materials including, but not limited to, collagen, fibrin and plant fibers. All of these materials, and others, may be used with the polymeric coatings described herein. Furthermore, the polymers can be used to fabricate an entire medical device.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface.

The epoxide-derived NO donating polymeric coatings can be applied to medical device surfaces, either primed or bare, in any manner known to those skilled in the art. Application methods include, but are not limited to, spraying, dipping, brushing, vacuum-deposition, electrostatic spray coating, plasma coating, spin coating electrochemical coating, and others. Moreover, the epoxide derived NO donating polymeric coatings may be used with a cap coat. A cap coat as used herein refers to the outermost coating layer applied over another coating. A NO donating polymer coating is applied over the primer coat. Then, a polymer cap coat is applied over the epoxide derived NO donating polymeric coating. The cap coat may optionally serve as a diffusion barrier to control the NO release. The cap coat may be merely a biocompatible polymer applied to the surface of the sent to protect the stent and have no effect on the NO release rates.

The epoxide-derived NO donating polymers are also useful for the delivery and controlled release of drugs. Drugs that are suitable for release from the polymers include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment the drugs controllably released include, but are not limited to, macrolide antibiotics including FKBP-12 binding agents. Exemplary drugs of this class include sirolimus (rapamycin) (Formula 18), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386) (Formula 17). Additionally, and other rapamycin hydroxyesters as disclosed in U.S. Pat. No. 5,362,718 may be used in combination with the polymers. The entire contents of all of preceding patents and patent applications are herein incorporated by reference for all they teach related to FKBP-12 binding compounds and the derivatives.

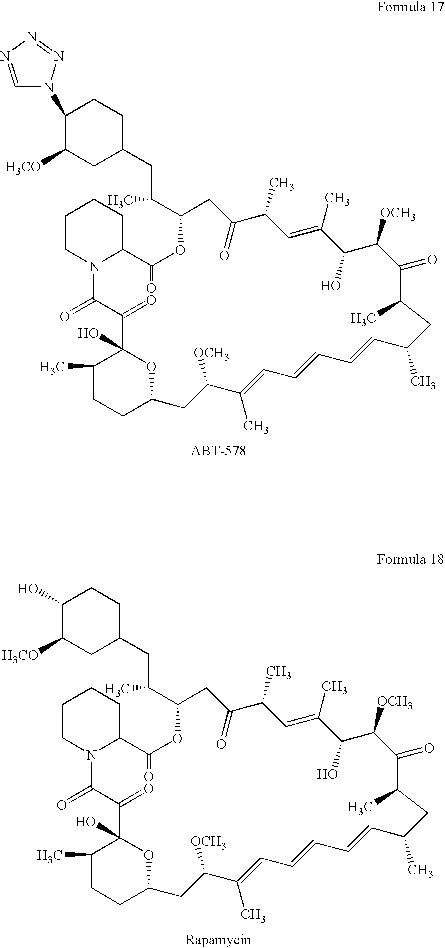

### EXAMPLES

The following non limiting examples provide methods for the synthesis of exemplary polymers described herein.

### Example 1

### Synthesis of the Polymer of Formula 5

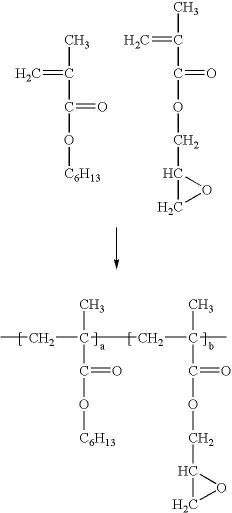

Glycidyl methacrylate (9.02 g), n-hexyl methacrylate (21.03 g), 1,4-dioxane (59.98 g) and of AIBN (240 mg) were mixed in a 120 mL bottle, which was sealed and purged with nitrogen for 30 minutes. The bottle was heated at 60° C. for 3 hours with stirring. The polymer was purified by repeated precipitation in methanol from dichloromethane solution. After drying in a vacuum oven at 45° C. overnight, a copolymer of with n-hexyl methacrylate (64 mol %) and glycidyl methacrylate (36 mol %) was obtained. The polymer has a number average molecular weight of 130,075 and PDI of 2.02 according to GPC (THF, 35C and polystyrene standards). The glass transition temperature of the polymer is 11° C.

### Example 2

### Conversion of the Epoxide Groups to Multiple Amine Groups in the Side Chains

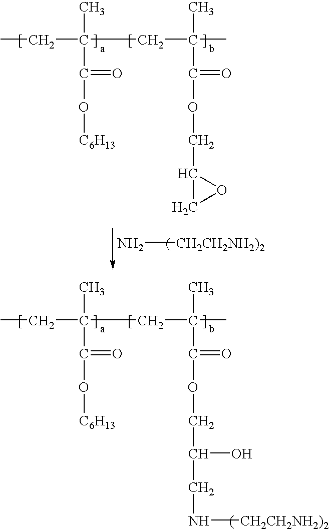

2.0 g of polymer from example 1 was dissolved in 8 mL THF. Separately another solution was prepared by mixing 23.9 mL of diethylenetriamine with 12 mL of THF. The polymer solution was added to the diethylenetriamine solution dropwise under agitation. The mixture was heated at 50° C. in an oil bath for three days. The resulting polymer was purified by precipitation into deionized water from THF solution. The ¹H NMR spectrum in d₄-methanol indicated the disappearance of the epoxide functional groups and the appearance of new peaks at around 2.7 ppm corresponding to the NCH₂ groups.

### Example 3

### Synthesizing a Secondary Amine Functionalized Dendrimer

A dendrimer with a surface of primary amine functional groups is reacted with 1,2-epoxypropane producing 2-hydroxypropylimine surface function groups on the dendrimer.

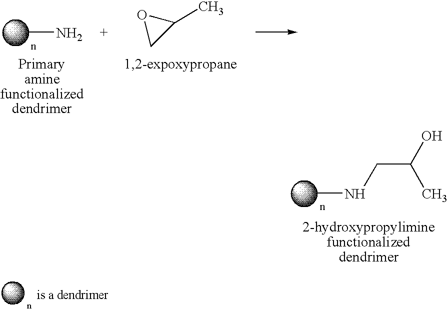

The 2-hydroxypropylimine can be reacted with the polymer of Example 1 to give a secondary amine functionalized dendrimer linked to the polymer.

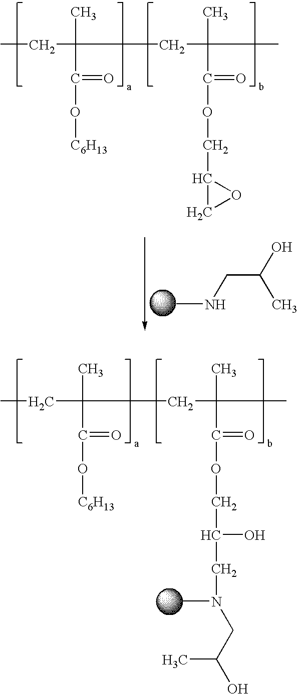

### Example 4

### General Method for the Ring Opening Reaction of Epoxides with Primary Amines

To a solution of Formula 4, 7 or 10 in THF is added R¹R²NH, wherein R¹ is hydrogen and a catalyst such as but not limited to LiClO₄, and the reaction stirred under anhydrous conditions. After the reaction has run to completion the THF is removed in vacuo and the solids washed with water. The polymers are then dried.

### Example 5

### Impregnation of Polymers with Copper

2.35 mg of CUCl₂. H₂O was dissolved in 1 mL of methanol. The methanol solution was slightly greenish. 3.40 mg of a polymer of the present invention was added to the methanol solution. The polymer was dissolved with agitation and the solution turned blue (indicating the chelation of the copper by the amine residues).

### Example 6

### Formation of Diazeniumdiolates

Polymers dissolved (typically 10 mg/50 mL) in THF are placed in a high pressure reaction vessel. At this step, one or more bioactive agents may be included in the polymer solution. An inert gas (including, but not limited to, argon and nitrogen) is then purged through the vessel. A base dissolved in a solvent (typically sodium methoxide or potassium methoxide in methanol) are then added in excess (typically 110% to 200%). Not all of the polymers of the present system require the use of a base in the NO loading process. For example, polymers containing functionalized dendrimers may not require the use of a base. The reaction is allowed to stir and the vessel purged with NO gas. The pressure of NO gas is increased (typically at least 15 psi) and the reaction mixture is then stirred further for at least 24 hours. At the end of the required time for the formation of diazeniumdiolates, dry hydrophobic solvents (typically hexanes or methyl t-butyl ether) are added to aid in the precipitation of the polymers. The polymers are then filtered and dried.

### Example 7

### Coating Implantable Vascular Stents

A 1% solution of a biodegradable NO-donating polymer and optionally a bioactive agent such as ABT-578 (in one embodiment in a polymer:drug ratio of 70:30 by weight), in chloroform is sprayed on a vascular stent and allowed to dry producing a controlled release coating on the vascular stent. Next the solubilized polymer (with or without added bioactive agents) is applied to the surfaces of an implantable medical device using methods known to those skilled in the art such as, but not limited to, rolling, dipping, spraying and painting. Excess polymer is removed under a gentle stream of warm inert gas such as, but not limited to argon or bone-dry nitrogen. The release of drug from the stent into a solvent is measured by high performance liquid chromatography (HPLC).

### Example 8

### Formation of Diazeniumdiolates on Polymer-Coated Vascular Stents

A vascular stent coated with at least one polymer from Examples 2 and 4 is placed in a 13 mm×100 mm glass test tube. Ten milliliters of 3% sodium methoxide in methanol or acetonitrile is added to the test tube, which is then placed in a 250 mL stainless steel Parr® apparatus. The apparatus is degassed by repeated cycles (×10) of pressurization/depressurization with nitrogen gas at 10 atmospheres. Next, the vessel undergoes 2 cycles of pressurization/depressurization with NO at 30 atmospheres. Finally, the vessel is filled with NO at 30 atmospheres and left at room temperature for 24 hrs. After 24 hrs, the vessel is purged of NO and pressurized/depressurized with repeated cycles (×10) of nitrogen gas at 10 atmospheres. The test tube is removed from the vessel and the 3% sodium methoxide solution is decanted. The stent is then washed with 10 mL of methanol (×1) and 10 mL of diethyl ether (×3). The stent is then removed from the test tube and dried under a stream of nitrogen gas. This procedure results in a diazeniumdiolated polymer-coated vascular stent.

### Example 9

### Manufacture of Stents from Epoxide-Derived NO-Donating Polymers

For exemplary, non-limiting, purposes a vascular stent will be described. A biodegradable NO-donating polymer is heated until molten in the barrel of an injection molding machine and forced into a stent mold under pressure. After the molded polymer (which now resembles and is a stent) is cooled and solidified the stent is removed from the mold. In one embodiment of the present invention the stent is a tubular shaped member having first and second ends and a walled surface disposed between the first and second ends. The walls are composed of extruded polymer monofilaments woven into a braid-like embodiment. In the second embodiment, the stent is injection molded or extruded. Fenestrations are molded, laser cut, die cut, or machined in the wall of the tube. In the braided stent embodiment monofilaments are fabricated from polymer materials that have been pelletized then dried. The dried polymer pellets are then extruded forming a coarse monofilament which is quenched. The extruded, quenched, crude monofilament is then drawn into a final monofilament with an average diameter from approximately 0.01 mm to 0.6 mm, preferably between approximately 0.05 mm and 0.15 mm. Approximately 10 to approximately 50 of the final monofilaments are then woven in a plaited fashion with a braid angle about 90 to 170 degrees on a braid mandrel sized appropriately for the application. The plaited stent is then removed from the braid mandrel and disposed onto an annealing mandrel having an outer diameter of equal to or less than the braid mandrel diameter and annealed at a temperature between about the polymer glass transition temperature and the melting temperature of the polymer blend for a time period between about five minutes and about 18 hours in air, an inert atmosphere or under vacuum. The stent is then allowed to cool and is then cut.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A nitric oxide (NO)-donating polymer comprising a diazeniumdiolate binding scaffolding;
said scaffolding comprising monomers with at least one diazeniumdiolate binding site;
wherein said binding sites comprise secondary amines.

2. The NO-donating polymer according to claim 1, further comprising multiple amine chelated copper ions.

3. The NO-donating polymer according to claim 1, wherein said polymer comprises at least one monomer selected from the group consisting of methyl methacrylate, ethyl methacrylate, propyl methacrylate, butyl methacrylate, pentyl methacrylate, hexyl methacrylate, 2-ethylhexyl methacrylate, octyl methacrylate, lauryl methacrylate, 2-ethoxyethyl methacrylate, 2-hydroxyethyl methacrylate, and hydroxypropyl methacrylate, methyl acrylate, ethyl acrylate, propyl acrylate, butyl acrylate, pentyl acrylate, hexyl acrylate, 2-ethylhexyl acrylate, octyl acrylate and lauryl acrylate, 2-hydroxyethyl acrylate, and hydroxypropyl acrylate. In one embodiment, the acrylate monomer is glycidyl methacrylate which has epoxide side chain. Non-acrylate monomers include, but are not limited to, ε-caprolactone, polyethylene glycol (PEG), trimethylene carbonate, lactide, glycolide, p-dioxanone, N-acetyl caprolactone, cyclohexyl caprolactone, 4-tert-butyl caprolactone, the caprolactone of Formula 4, and their derivatives and combinations thereof.

4. The NO-donating polymer according to claim 1, wherein said polymer comprises the coating of an implantable medical device.

5. The NO-donating polymer according to claim 1, wherein said polymer comprises an implantable medical device.

6. The NO-donating polymer according to claim 4 or 5, wherein said medical device is selected from the group consisting of vascular stents, stent grafts, urethral stents, bile duct stents, catheters, guide wires, pacemaker leads, bone screws, sutures and prosthetic heart valves.

7. The NO-donating polymer according to claim 1 wherein said polymer comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

8. The NO-donating polymer according to claim 1, wherein said secondary amines are on a dendrimer.

9. The NO-donating polymer according to claim 1, wherein said secondary amines can be coordinated with a copper ligand.

10. A medical device comprising a diazeniumdiolate binding scaffolding;
a polymer comprises said scaffolding having monomers with at least one diazeniumdiolate binding site;
wherein said binding sites comprise secondary amines; and
wherein a medical device comprises said scaffolding.

11. The medical device according to claim 10, further comprising multiple amine chelated copper ions.

12. The medical device according to claim 10, wherein said polymer comprises at said polymer comprises at least one monomer selected from the group consisting of methyl methacrylate, ethyl methacrylate, propyl methacrylate, butyl methacrylate, pentyl methacrylate, hexyl methacrylate, 2-ethylhexyl methacrylate, octyl methacrylate, lauryl methacrylate, 2-ethoxyethyl methacrylate, 2-hydroxyethyl methacrylate and hydroxypropyl methacrylate, methyl acrylate, ethyl acrylate, propyl acrylate, butyl acrylate, pentyl acrylate, hexyl acrylate, 2-ethylhexyl acrylate, octyl acrylate and lauryl acrylate, 2-hydroxyethyl acrylate, and hydroxypropyl acrylate. In one embodiment, the acrylate monomer is glycidyl methacrylate which has epoxide side chains. Non-acrylate monomers include, but are not limited to, ε-caprolactone, polyethylene glycol (PEG), trimethylene carbonate, lactide, glycolide, p-dioxanone, N-acetyl caprolactone, cyclohexyl caprolactone, 4-tert-butyl caprolactone, the caprolactone of Formula 4, and their derivatives and combinations thereof.

13. The medical device according to claim 10, wherein said polymer comprises the coating of an implantable medical device.

14. The medical device according to claim 10, wherein said polymer comprises an implantable medical device.

15. The medical device according to claim 13 or 14, wherein said medical device is selected from the group consisting of vascular stents, stent grafts, urethral stents, bile duct stents, catheters, guide wires, pacemaker leads, bone screws, sutures and prosthetic heart valves.

16. The medical device according to claim 10, wherein said polymer comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

17. The medical device according to claim 10, wherein said secondary amines are on a dendrimer.

18. The medical device according to claim 10, wherein said secondary amines can be coordinated with a copper ligand.

19. A method of forming a NO-donating polymer comprising a NO-donating scaffolding comprising:
a) providing a polymer with side chains having at least one epoxide;
b) reacting an amine with said epoxide wherein said amine comprises a NO-donating scaffolding thereby forming a NO-donating scaffolded polymer; and
c) loading said scaffolded polymer with NO thereby forming an NO-donating polymer.

20. A method of forming a NO-donating polymer according to claim 19, wherein said method further comprises the step of:
d) forming at least a portion of a medical device with said NO-donating polymer.

21. The amine from claim 19 b) is selected from N-methylethylenediamine, N-methylpropylylenediamine, N-methylbutylenediamine, N-ethylethylenediamine, N-ethylpropylylenediamine, N-ethylbutylenediamine, N-benzylethylenediamine, N-benzylpropylylenediamine, N-benzylbutylenediamine, N-propylethylenediamine, N-propylpropylylenediamine, N-propylbutylenediamine, ethylenediamine, diethylenetriamine, triethylenetetramine, tetraethylenepentaamine, pentaethylenehexamine, and hexaethyleneheptaamine.
